(19) United States
(12) Patent Application Publication
(10) Pub. No.: US 2016/0158458 A1
(43) Pub. Date: Jun. 9, 2016

(54) Safe Injection Device Capable of Locking Needle

(71) Applicant: WUXI YUSHOU MEDICAL APPLIANCES Co., LTD., Wuxi City (CN)

(72) Inventors: Zhong FENG, Wuxi City (CN); Zhiling FENG, Wuxi City (CN); Chunyin SU, Wuxi City (CN); Liping MIAO, Wuxi City (CN); Ping XU, Wuxi City (CN); Jingjuan ZHU, Wuxi City (CN); Yunfei GU, Wuxi City (CN)

(21) Appl. No.: 14/562,765

(22) Filed: Dec. 7, 2014

(51) Int. Cl. A61M 5/32

(52) U.S. Cl. CPC A61M 5/3216

(57) ABSTRACT

An injection needle assembly includes a needle extended from a needle seat and a lockable needle arrangement which includes a locking platform radially extended from the needle seat, a needle enclosure foldably extended from the locking platform for enclosing the needle, and a locker unit including a first locker provided at the locking platform and a second locker provided at the needle enclosure, wherein the first and second lockers are engaged in a non-releasable manner to securely lock up the needle enclosure on the locking platform for ensuring the needle to be enclosed within the needle enclosure. Therefore, after the use of the needle for injection, the needle is enclosed and protected by the needle enclosure for preventing any cross infection.

### NOTICE OF COPYRIGHT

A portion of the disclosure of this patent document contains material which is subject to copyright protection. The copyright owner has no objection to any reproduction by anyone of the patent disclosure, as it appears in the United States Patent and Trademark Office patent files or records, but otherwise reserves all copyright rights whatsoever.

### BACKGROUND OF THE PRESENT INVENTION

1. Field of Invention

The present invention relates to an injection device, and more particularly relates to an injection needle assembly with lockable needle arrangement which can be used to lock up the used injection needle to prevent cross-infection.

2. Description of Related Arts

Disposable syringes are widely used in medical area for avoiding cross-infection of diseases in those years. In order to prevent the disposable syringes being reused, self destroying structures are developed for the syringe. However, even though the self destroying structure can reduce the chance of cross-infection of diseases owing to its self destroy structure, it still has many drawbacks. The exposed needles of the used syringes still cause a lot of trouble during the collecting, transporting, and disposing. Moreover, the people dealing with the used syringes still have the risk of being injured.

Accordingly, improper management of used needles can pose a health risk to public and waste workers. For example, discarding the used needles without any protection may potentially cause infection when the syringes are broken accidentally. Janitors and housekeepers also risk injury if loose sharps poke through plastic garbage bags. Used needles can transmit serious diseases, such as HIV and hepatitis.

In order to provide a safety feature of the syringe, the self destroying structure generally comprises a compression spring supported in the barrel. Therefore, after the medication is injected through the needle, the spring retracts the needle into the barrel. However, the structure of the self destroying structure is relatively complicated and hard to assemble during manufacturing. An unintentional pushing force at the syringe will cause the actuation of the spring to retract the needle into the barrel especially during injection. Furthermore, it is hard to remove the used needle in the barrel the used syringe is collected.

Another type of self destroying structure for the syringe comprises an elongated lock sleeve to enclose the used needle after injection. However, the lock sleeve can be detached from the syringe by any excessive force. In other words, the used needle may be exposed once the lock sleeve is loosen or even detached from the syringe. Furthermore, the locking mechanism of the lock sleeve must be precisely matched with the syringe. Therefore, the manufacturing cost of the lock sleeve will be relatively high and the manufacturing process of the lock sleeve will be relatively complicated. In addition, the lock sleeve can only be designed for one particular syringe with the corresponding structure. In other words, the lock sleeve is not a universal locking device to incorporate different types or sizes of the syringes.

To solve the mentioned deficiencies, the present invention provides a new design of an injection device with needle lock device which has some advantages. For example, the new design makes the molding process easier and the production cost lower wherein the production efficiency is improved, and at the same time, the needle is not easy to be unlocked.

### SUMMARY OF THE PRESENT INVENTION

The invention is advantageous in that it provides an injection needle assembly with lockable needle arrangement which can be used to lock up the used injection needle to prevent cross-infection.

Another advantage of the invention is to provide a lockable needle arrangement, which can incorporate with any injection needle assembly to lock up the used injection needle.

Another advantage of the invention is to provide a lockable needle arrangement, which provides a safe and convenient way to lock the used injection needle while being cost effective.

Another advantage of the invention is to provide a lockable needle arrangement, which does not involve complicated and expensive mechanical components and processes so that the manufacturing cost of the present invention can be minimized.

Another advantage of the invention is to provide a lockable needle arrangement, which simplifies the manufacturing process to enhance the mass production of the lockable needle arrangement for the injection apparatus.

Another advantage of the invention is to a lockable needle arrangement, wherein no expensive or complicated structure is required to employ in the present invention in order to achieve the above mentioned objects. Therefore, the present invention successfully provides an economic and efficient solution for protecting a person from being hurt by the used needle and for ensuring the used needle to be reused.

Additional advantages and features of the invention will become apparent from the description which follows, and may be realized by means of the instrumentalities and combinations particular point out in the appended claims.

According to the present invention, the foregoing and other objects and advantages are attained by a lockable needle arrangement for an injection needle assembly which comprises a needle extended from a needle seat, comprising:

a locking platform arranged for extending from the needle seat;

a needle enclosure, having a U-shaped cross-section, foldably extended from the locking platform for enclosing the needle; and

a locker unit comprising a first locker provided at the locking platform and a second locker provided at the needle enclosure, wherein the first and second lockers are engaged in a non-releasable manner to securely lock up the needle enclosure on the locking platform for ensuring the needle to be enclosed within the needle enclosure.

Accordingly, the lockable needle arrangement can be configured to incorporate with any existing injection needle having the needle seat.

In accordance with another aspect of the invention, the present invention comprises an injection needle assembly, comprising a needle upwardly extended from a needle seat and a lockable needle arrangement. The lockable needle arrangement comprises:

a locking platform radially extended from the needle seat;

a needle enclosure, having a U-shaped cross-section, foldably extended from the locking platform to form an elongated side opening for enclosing the needle; and

a locker unit comprising a first locker provided at the locking platform and a second locker provided at the needle enclosure, wherein the first and second lockers are engaged in a non-releasable manner to securely lock up the needle enclosure on the locking platform for ensuring the needle to be enclosed within the needle enclosure.

Accordingly, the lockable needle arrangement can be integrated with the needle seat to form an integrated needle assembly for any existing syringe.

Still further objects and advantages will become apparent from a consideration of the ensuing description and drawings.

These and other objectives, features, and advantages of the present invention will become apparent from the following detailed description, the accompanying drawings, and the appended claims.

### BRIEF DESCRIPTION OF THE DRAWINGS

FIG. 1 is a perspective view of an injection needle assembly with a lockable needle arrangement according to a preferred embodiment of the present invention.

FIG. 2 is a partially sectional view of the injection needle assembly with the lockable needle arrangement according to the above preferred embodiment of the present invention, illustrating the needle enclosure being unfolded to expose the needle.

FIG. 3 is a sectional view of the injection needle assembly with the lockable needle arrangement according to the above preferred embodiment of the present invention, illustrating the needle enclosure being folded to enclose the needle.

FIG. 4 is a perspective view of the lockable needle arrangement according to the above preferred embodiment of the present invention.

FIG. 5 is a sectional view of an enclosure body of the lockable needle arrangement according to the above preferred embodiment of the present invention, illustrating the needle cap received at the enlarged cap cavity.

FIG. 6 is a sectional view of an enclosure body of the lockable needle arrangement according to the above preferred embodiment of the present invention, illustrating the needle received at the narrowed needle cavity.

FIG. 7 is a sectional view of a locker unit of the lockable needle arrangement according to the above preferred embodiment of the present invention.

FIG. 8 illustrates an alternative mode of the locker unit of the lockable needle arrangement according to the above preferred embodiment of the present invention.

FIG. 9 illustrates an alternative mode of the locking platform of the lockable needle arrangement according to the above preferred embodiment of the present invention, illustrating the integrated structure of the locking platform with the needle seat.

FIG. 10 illustrates an alternative mode of the enclosure body of the lockable needle arrangement according to the above preferred embodiment of the present invention.

### DETAILED DESCRIPTION OF THE PREFERRED EMBODIMENT

The following description is disclosed to enable any person skilled in the art to make and use the present invention. Preferred embodiments are provided in the following description only as examples and modifications will be apparent to those skilled in the art. The general principles defined in the following description would be applied to other embodiments, alternatives, modifications, equivalents, and applications without departing from the spirit and scope of the present invention.

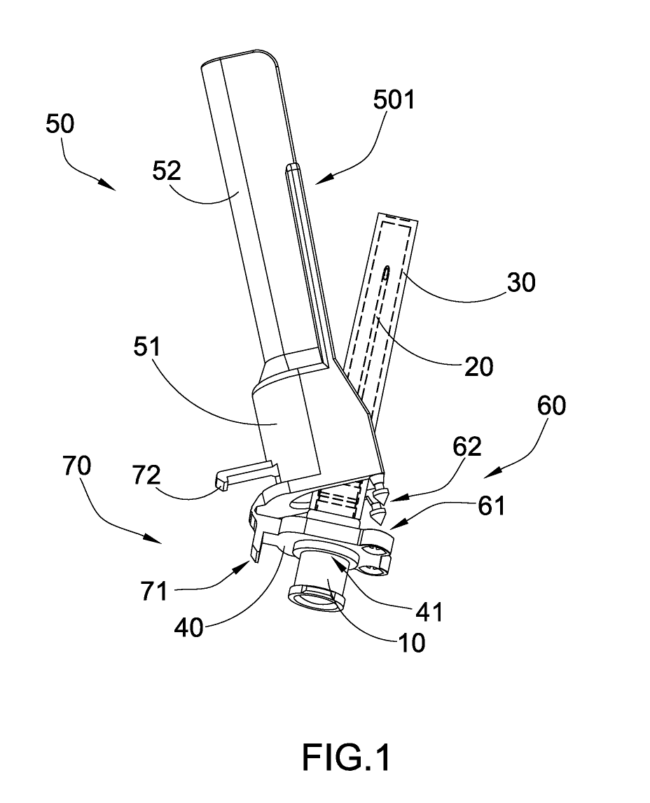

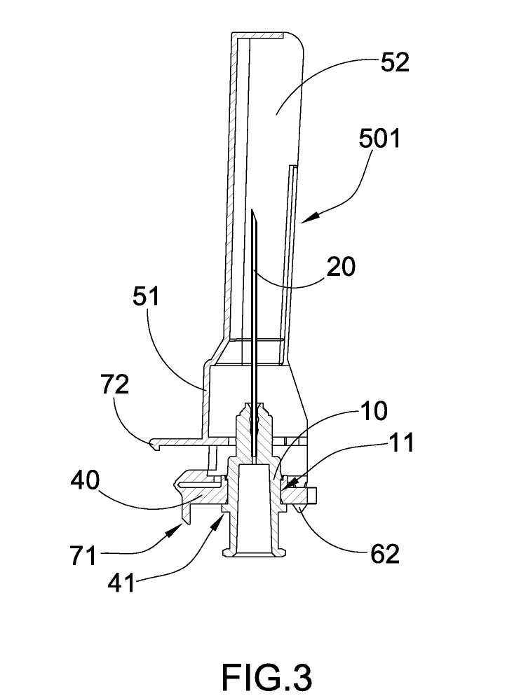

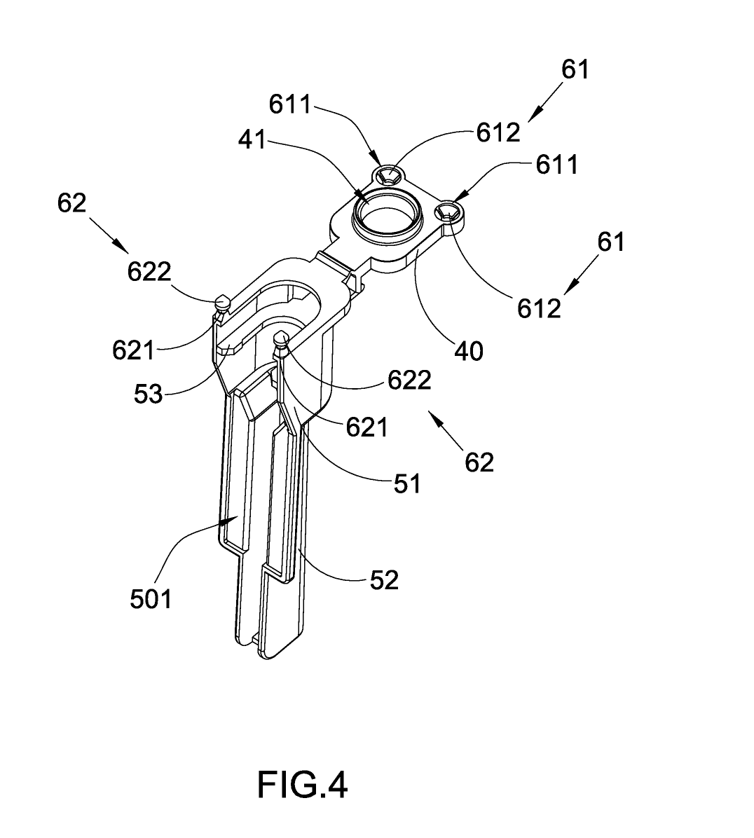

Referring to FIGS. 1 to 4 of the drawings, an injection needle assembly according to a preferred embodiment of the present invention is illustrated, wherein the injection needle assembly comprises a needle seat 10 and a needle 20, i.e. an injection needle, upwardly extended from the needle seat 10. Accordingly, the needle seat 10 can be coupled with a syringe for injecting medication fluid through the needle 20.

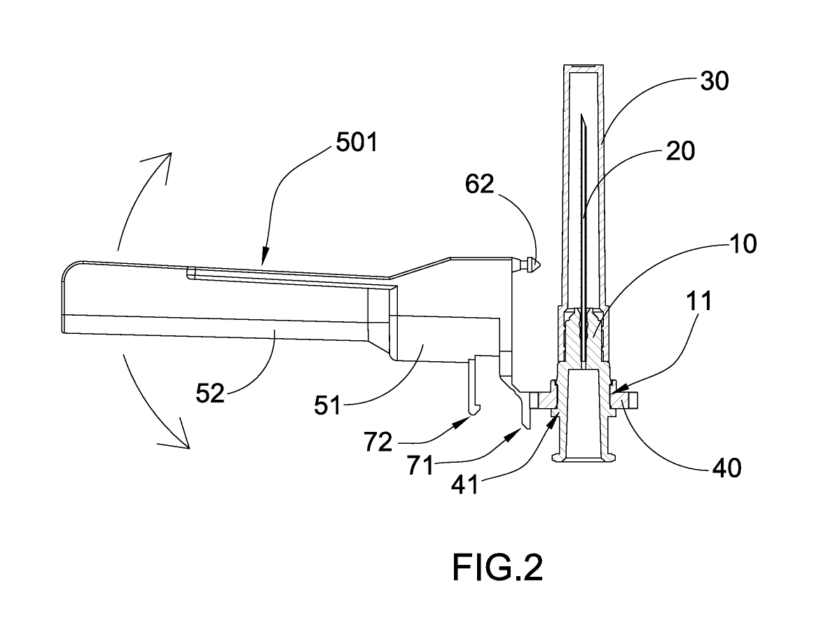

The injection needle assembly further comprises an elongated needle cap 30 detachably coupled at the needle seat 10 to enclose the needle 20. Before the needle 20 is used for injection, the needle 20 is enclosed and protected by the needle cap 30 to maintain the sterilized needle 20 in a sanitation and hygiene condition. As shown in FIG. 2, the needle seat 10 has an upper portion and a lower portion having a diameter larger than that of the upper portion, wherein an opening of the needle cap 30 is detachably coupled at the upper portion of the needle seat 10. In other words, the diameter of the opening of the needle cap 30 is slightly larger than the diameter of the upper portion of the needle seat 10. Preferably, the diameter of the opening of the needle cap 30 matches with the diameter of the lower portion of the needle seat 10.

The injection needle assembly further comprises a lockable needle arrangement for locking the needle 20 after injection. The lockable needle arrangement comprises a locking platform 40, a needle enclosure 50, and a locker unit 60.

As shown in FIGS. 1 to 4, the locking platform 40 is radially extended from the needle seat 10. In particular, the locking platform 40 is extended from the lower portion of the needle seat 10. Accordingly, the locking platform 40 has a retention slot 41, wherein the needle seat 10 is coupled at the retention slot 41 at a position that the retention slot 41 is engaged with the circumferential surface of the needle seat 10 at the lower portion thereof. Therefore, the diameter of the opening of the needle cap 20 is smaller than the diameter of the retention slot 41 of the locking platform 40. Preferably, the needle seat 10 has an engaging slot 11 indented at the circumferential surface at the lower portion, wherein the retention slot 41 is engaged with the engaging slot 11 to retain the locking platform 40 at the needle seat 10. Furthermore, the locking platform 40 further has a first extended portion extended from one side of the retention slot 41 and an opposed extended portion extended from the opposite side of the retention slot 41.

The needle enclosure 50 is foldably extended from the locking platform 40 to for enclosing the needle 20. Accordingly, the needle enclosure 50 is pivotally folded between an unfolded position to expose the needle 20 and a folded position to enclose the needle 20. In particular, the needle enclosure 50 has a U-shaped cross section to form an elongated side opening 501 for enclosing the needle 20 therethrough. According to the preferred embodiment, the needle enclosure 50 comprises an enclosure base 51 foldably and integrally extended from the locking platform 40 and an enclosure body 52 integrally extended from the enclosure base 51.

The enclosure base 51 has a folding edge portion foldably and integrally extended from an edge of the first extended portion of the locking platform 40 and a locking edge portion where the locker unit 60 is provided thereat. Preferably, the size and shape of the enclosure base 51 matches with the size and shape of the locking platform 40, such that the enclosure base 51 can be fittedly supported by the locking platform 40. In addition, the bottom side of the enclosure base 51 is a flat surface while the top side of the locking platform 40 is also a flat surface to support the bottom side of the enclosure base 51.

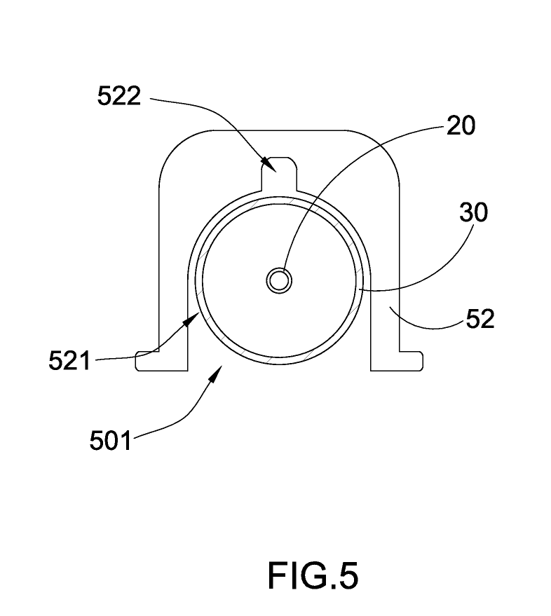

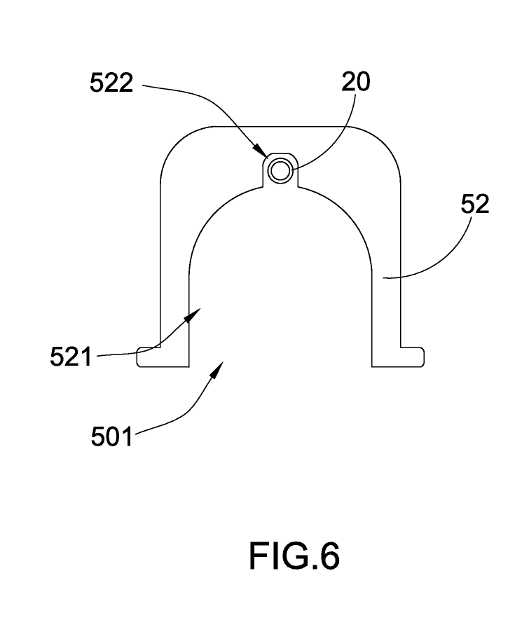

Accordingly, the length of the enclosure body 52 is longer than the length of the needle 20 and is longer than the length of the needle cap 30. The enclosure body 52 has an enlarged cap cavity 521 formed at the side opening 501 for receiving the needle cap 30, as shown in FIG. 5, and a narrowed needle cavity 522 aligned with the cap cavity 521 to receive the needle 20, such that after the needle cap 30 is detached from the needle seat 10, the needle enclosure 50 is folded to enclose the needle 20 within the needle cavity 522 through the cap cavity 521, as shown in FIG. 6. In other words, the cap cavity 521 is located between the needle cavity 522 and the side opening 501, such that the needle cavity 522 is located away from the side opening 501 of the needle enclosure 50.

Accordingly, the size of the cap cavity 521 is slightly larger than the diameter of the needle cap 30 such that the needle cap 30 can be received in the cap cavity 521. In addition, the size of the needle cavity 522 is smaller than that of the cap cavity 521 and is larger than the diameter of the needle 20. In other words, only the needle 20 can received in the needle cavity 522 while the needle cap 30 cannot receive in the needle cavity 522.

Preferably, the enclosure body 52 has a trapezoid shape that a width of the enclosure body 52 is gradually reduced toward the enclosure base 51. Therefore, the enclosure body 52 is able to enclose the needle 20 with the needle cap 30 within the cap cavity 521, as shown in FIG. 5, when the needle cap 30 is mounted to the needle seat 10 and is able to enclose the needle 20 without the needle cap 30 within the needle cavity 522, as shown in FIG. 6, when the needle cap 30 is removed from the needle seat 10.

As shown in FIG. 2, the needle enclosure 50 further comprises a U-shaped holding rim 53 integrally protruded from an inner wall of the enclosure base 51 to hold the needle cap 30 within the cap cavity 521 when the needle 20 is enclosed within the needle cap 30.

As shown in FIGS. 1 to 4, the locker unit 60 comprises a first locker 61 provided at the locking platform 40 and a second locker 62 provided at the needle enclosure 50, wherein the first and second lockers 61, 62 are engaged in a non-releasable manner to securely lock up the needle enclosure 50 on the locking platform 40 for ensuring the needle 20 to be enclosed within the needle enclosure 50.

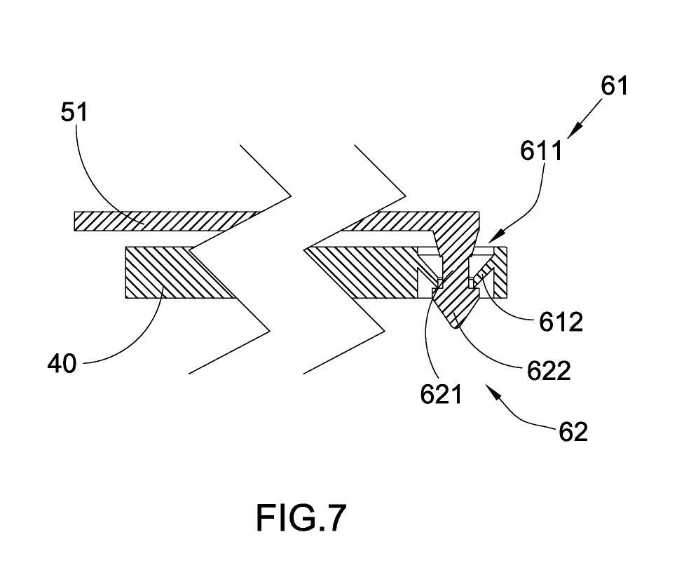

According to the preferred embodiment, the first locker 61, preferably two first lockers, is integrally formed at the second extended portion of the locking platform 40. In particular, the first locker 61 has at least a locking slot 611 formed at the locking platform and a plurality of locking flaps 612 radially extended from a circumferential wall of the locking slot 611 toward a center thereof, such that when the second locker 62 is extended through the locking slot 611, the locking flaps 612 are engaged with the second locker 62 to prevent the second locker 62 being detached from the locking slot 611, as shown in FIG. 7. Preferably, the locking flaps 612 are made of flexible material to ensure the insertion of the second locker 62 in one direction.

The second locker 62 has an elongated neck portion 621 integrally extended from the enclosure base 51 at the locking edge portion thereof, and an enlarged head portion 622 extended from the neck portion 621, such that when the head portion 611 of the second locker 62 passes through the locking slot 611, the locking flaps 612 are engaged with the neck portion 621 of the second locker 62, as shown in FIG. 7. Preferably, the head portion 622 of the second locker 62 has a conical shape. Preferably, two second lockers 62 are spacedly extended from the needle enclosure 50 to couple with two first lockers 61 at the locking platform 40 respectively.

It is worth mentioning that when the needle cap 30 is enclosed within the cap cavity 521, the second locker 62 is not engaged with the first locker 61, such that the needle enclosure 50 can be pivotally unfolded to expose the needle cap 30. Once the needle cap 30 is detached from the needle seat 10, the needle enclosure 50 can be pivotally folded to enclose the needle 20 within the needle cavity 522. The first and second lockers 61, 62 are engaged with each other only when the needle 20 is enclosed within the needle cavity 522.

The injection needle assembly further comprises a retention unit 70 for retaining the needle enclosure in the unfolded position, wherein the retention unit 70 has a retention edge 71 formed at the locking platform 40 and a retention arm 72 integrally extended from the needle enclosure 50, such that when the retention arm 72 is detachably engaged with the retention edge 71, the needle enclosure 50 is unfolded from the locking platform 40 for exposing the needle 20.

Accordingly, the retention edge 71 is integrally formed at the edge of the first extended portion of the locking platform 40 where the folding edge portion of the enclosure base 51 is extended. The retention arm 72, having a hooking free end, is integrally extended from the enclosure base 51. The needle enclosure 50 is pivotally and downwardly folded to engage the hooking free end of the retention arm 72 with the retention edge 71, so as to retain the needle enclosure 50 parallel to the needle 20.

In order to operate the injection needle assembly of the present invention for preparing an injection, the operator, such as a physician, is able to unfold the needle enclosure 50 by pivotally and downwardly moving the needle enclosure 50 from the locking platform 40 so as to move the needle cap 30 out of the cap cavity 521 of the needle enclosure 50. Accordingly, the needle enclosure 50 is pivotally folded until the retention arm 72 is engaged with the retention edge 71 to retain the needle enclosure 50 in position. Then, the needle cap 30 can be removed from the needle seat 10 to expose the needle 20. The operator is able to extract the fluid medicine through the needle 20 as the normal operation of the syringe. After the injection, the needle enclosure 50 can be pivotally and upwardly folded back to enclose the needle 20 within the needle enclosure 50 through the side opening 501 thereof until the second locker 62 is engaged with the first locker 61. It is worth mentioning that the operator does not require putting the needle cap 30 back to enclose the used needle 20 after injection. The needle 20 will be received within the needle cavity 522 of the needle enclosure 50 and will be locked therewithin once the first and second lockers 61, 62 are engaged with each other. It is worth mentioning that the needle enclosure 50 cannot be unfolded to expose the used needle 20 after the first and second lockers 61, 62 are engaged with each other.

It is worth mentioning that the locking platform 40, the needle enclosure 50, and the locker unit 60 are integrally formed in a one-piece integrated member preferably by mold injection, such that the lockable needle arrangement of the present invention can be mass produced to incorporate with any injection needle assembly having the needle seat 10. It is worth mentioning that since the diameter of the retention slot 41 of the locking platform 40 is larger than the diameter of the needle cap 30, any existing injection needle assembly can be incorporated with the lockable needle arrangement of the present invention by slidably inserting the needle cap 30 through the retention slot 41 until the locking platform 40 is coupled at the needle seat 10. In other words, the operator is able to couple the locking platform 40 to the conventional needle seat of the injection needle assembly without removing the conventional needle cap so as to keep the needle to be enclosed within the needle cap during the assembling operation of the lockable needle arrangement of the present invention.

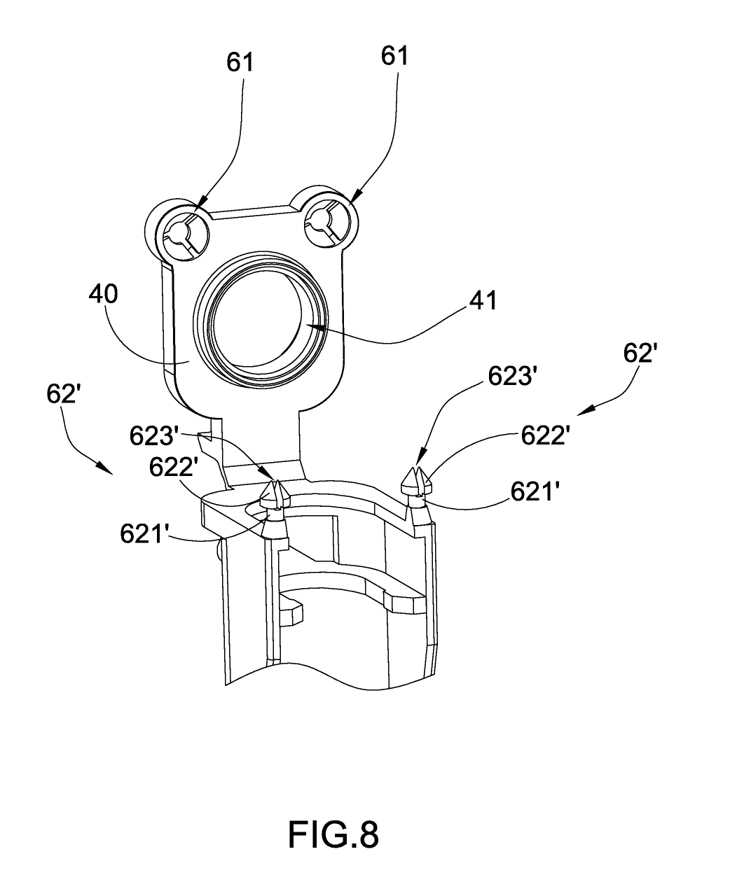

FIG. 8 illustrates an alternative mode of the second locker 62′ which is a modification of the above embodiment, wherein the second locker 62′ further has a slit 623′ extended from the head portion 622′ to the neck portion 621′ so as to split the second locker 62′ into two locking portions for enhancing the elasticity of the second locker 62′ to engage with the first locker 61.

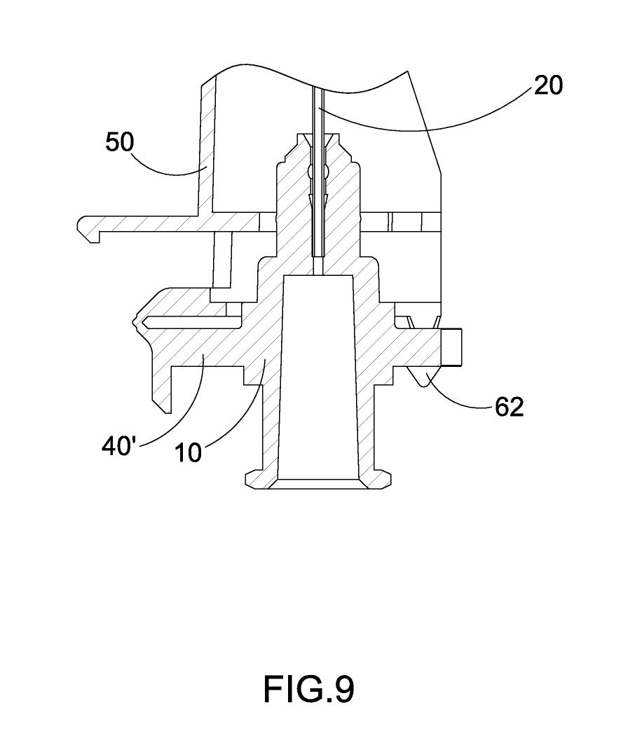

FIG. 9 illustrates an alternative mode of the locking platform 40′ of the lockable needle arrangement, wherein the locking platform 40′ is integrally extended from the needle seat 10 to form a one piece integrated structure. Therefore, the lockable needle arrangement is integrally built-in with the injection needle assembly. The injection needle assembly with the built-in the lockable needle arrangement can be made by mold injection and can be incorporated with any existing syringe.

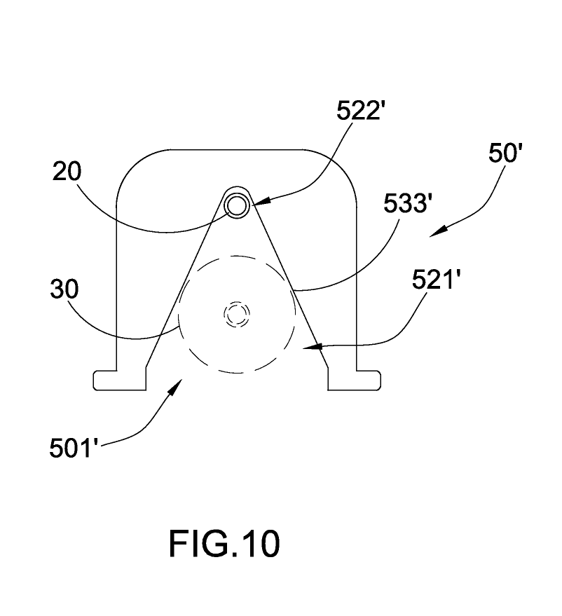

FIG. 10 illustrates an alternative mode of the enclosure body 50′ of the lockable needle arrangement to form the cap cavity 521′ and the needle cavity 522′ between two inner sidewalls 533′. Accordingly, the two sidewalls 533′ of the enclosure body 50′ are two slanted sidewalls respectively, wherein a distance between the sidewalls 533′ is gradually increasing toward the side opening 501′, such that the size of the cap cavity 521′ is larger than the size of the needle cavity 522′.

One skilled in the art will understand that the embodiment of the present invention as shown in the drawings and described above is exemplary only and not intended to be limiting.

It will thus be seen that the objects of the present invention have been fully and effectively accomplished. The embodiments have been shown and described for the purposes of illustrating the functional and structural principles of the present invention and is subject to change without departure from such principles. Therefore, this invention includes all modifications encompassed within the spirit and scope of the following claims.

What is claimed is:

1. A lockable needle arrangement for an injection needle assembly which comprises a needle extended from a needle seat, comprising:
a locking platform arranged for extending from said needle seat;
a needle enclosure, having a U-shaped cross-section, foldably extended from said locking platform for enclosing said needle; and
a locker unit comprising a first locker provided at said locking platform and a second locker provided at said needle enclosure, wherein said first and second lockers are engaged in a non-releasable manner to securely lock up said needle enclosure on said locking platform for ensuring said needle to be enclosed within said needle enclosure.

2. The lockable needle arrangement, as recited in claim 1, wherein said first locker has at least a locking slot formed at said locking platform and a plurality of locking flaps radially extended from a circumferential wall of said locking slot toward a center thereof, such that when said second locker is extended through said locking slot, said locking flaps are engaged with said second locker to prevent said second locker being detached from said locking slot.

3. The lockable needle arrangement, as recited in claim 2, wherein said second locker has an elongated neck portion extended from said needle enclosure and an enlarged head portion extended from said neck portion, such that when said head portion of said second locker passes through said locking slot, said locking flaps are engaged with said neck portion of said second locker.

4. The lockable needle arrangement, as recited in claim 3, wherein said needle enclosure comprises an enclosure base having a folding edge portion foldably and integrally extended from an edge of said locking platform and a locking edge portion where said second locker is provided thereat, and an enclosure body integrally extended from said enclosure base for enclosing said needle when said first and second lockers are engaged with each other.

5. The lockable needle arrangement, as recited in claim 4, wherein said locking platform has a retention slot for coupling with a circumferential surface of said needle seat so as to retain said locking platform at said needle seat.

6. The lockable needle arrangement, as recited in claim 5, further comprising a retention unit for retaining said needle enclosure in an unfolded position, wherein said retention unit has a retention edge formed at said locking platform and a retention arm integrally extended from said needle enclosure, such that when said retention arm is detachably engaged with said retention edge, said needle enclosure is unfolded from said locking platform for exposing said needle.

7. An injection needle assembly, comprising:
a needle seat;
a needle upwardly extended from said needle seat; and
a lockable needle arrangement which comprises:
a locking platform radially extended from said needle seat;
a needle enclosure, having a U-shaped cross-section, foldably extended from said locking platform to form an elongated side opening for enclosing said needle; and
a locker unit comprising a first locker provided at said locking platform and a second locker provided at said needle enclosure, wherein said first and second lockers are engaged in a non-releasable manner to securely lock up said needle enclosure on said locking platform for ensuring said needle to be enclosed within said needle enclosure.

8. The injection needle assembly, as recited in claim 7, further comprising an elongated needle cap detachably coupled at said needle seat to enclose said needle, wherein said needle cap is received within said needle enclosure before said needle is used.

9. The injection needle assembly, as recited in claim 8, wherein said needle enclosure comprises an enclosure base foldably and integrally extended from said locking platform and an enclosure body integrally extended from said enclosure base, wherein said enclosure body has an enlarged cap cavity formed at said side opening for receiving said needle cap and a narrowed needle cavity aligned with said cap cavity to receive said needle, such that after said needle cap is detached from said needle seat, said needle enclosure is folded to enclose said needle within said needle cavity through said cap cavity.

10. The injection needle assembly, as recited in claim 9, wherein said first and second lockers are engaged with each other only when said needle is enclosed within said needle cavity.

11. The injection needle assembly, as recited in claim 9, wherein said needle enclosure further comprises a U-shaped holding rim integrally protruded from an inner wall of said enclosure base to hold said needle cap within said cap cavity when said needle is enclosed within said needle cap.

12. The injection needle assembly, as recited in claim 10, wherein said enclosure base has a folding edge portion foldably and integrally extended from an edge of said locking platform and a locking edge portion where said second locker is provided thereat.

13. The injection needle assembly, as recited in claim 7, wherein said first locker has at least a locking slot formed at said locking platform and a plurality of locking flaps radially extended from a circumferential wall of said locking slot toward a center thereof, such that when said second locker is extended through said locking slot, said locking flaps are engaged with said second locker to prevent said second locker being detached from said locking slot.

14. The injection needle assembly, as recited in claim 12, wherein said first locker has at least a locking slot formed at said locking platform and a plurality of locking flaps radially extended from a circumferential wall of said locking slot toward a center thereof, such that when said second locker is extended through said locking slot, said locking flaps are engaged with said second locker to prevent said second locker being detached from said locking slot.

15. The injection needle assembly, as recited in claim 13, wherein said second locker has an elongated neck portion extended from said needle enclosure and an enlarged head portion extended from said neck portion, such that when said head portion of said second locker passes through said locking slot, said locking flaps are engaged with said neck portion of said second locker.

16. The injection needle assembly, as recited in claim 14, wherein said second locker has an elongated neck portion extended from said needle enclosure and an enlarged head portion extended from said neck portion, such that when said head portion of said second locker passes through said locking slot, said locking flaps are engaged with said neck portion of said second locker.

17. The injection needle assembly, as recited in claim 7, further comprising a retention unit for retaining said needle enclosure in an unfolded position, wherein said retention unit has a retention edge formed at said locking platform and a retention arm integrally extended from said needle enclosure, such that when said retention arm is detachably engaged with said retention edge, said needle enclosure is unfolded from said locking platform for exposing said needle.

18. The injection needle assembly, as recited in claim 16, further comprising a retention unit for retaining said needle enclosure in an unfolded position, wherein said retention unit has a retention groove formed at said locking platform and a retention arm integrally extended from said needle enclosure, such that when said retention arm is detachably engaged with said retention groove, said needle enclosure is unfolded from said locking platform for exposing said needle.

19. The injection needle assembly, as recited in claim 7, wherein said locking platform has a retention slot coupled with an engaging slot of said needle seat to retain said locking platform at said needle seat.

20. The injection needle assembly, as recited in claim 7, wherein said locking platform is integrally extended from said needle seat.
